(19) United States
(12) Patent Application Publication
(10) Pub. No.: US 2018/0015191 A1
(43) Pub. Date: Jan. 18, 2018

(54) DEVICE AND METHOD FOR STERILIZING THERMOPLASTIC CONTAINERS USING A PULSED ELECTRON BEAM AND A MOBILE REFLECTOR

(71) Applicant: SIDEL PARTICIPATIONS, Octeville-sur-mer (FR)

(72) Inventors: Cedric BIANCHINI, Octeville-sur-mer (FR); Guy FEUILLOLEY, Octeville-sur-mer (FR)

(21) Appl. No.: 15/546,966

(22) PCT Filed: Jan. 20, 2016
(86) PCT No.: PCT/FR2016/050099

§ 371 Date: Jul. 27, 2017

(30) Foreign Application Priority Data
Jan. 28, 2015 (FR) 1550638

(51) Int. Cl. A61L 2/08; B67C 3/22

(52) U.S. Cl. CPC A61L 2/087; B67C 3/22; A61L 2202/23; B67C 2003/228

(57) ABSTRACT

Disclosed is a device and a method for sterilizing thermoplastic containers using a pulsed beam of electrons which is formed of a succession of pulses each having an emission time which is less than 100 ns and an intensity which is greater than 1 kA so as to sterilize, through a wall of the container, at least the inside of the container. A reflector mounted with the ability to move axially relative to the container is included.

This invention relates to a device and a method for sterilizing thermoplastic containers using a pulsed electron beam.

Various sterilization methods for sterilizing at least the inside of a preform and/or a preform made of thermoplastic material are known from the state of the art.

The manufacturing of a container made of thermoplastic material is achieved starting from a hot preform, in general thermally conditioned in advance in a furnace of a container-manufacturing plant before being inserted into a mold to be transformed therein by blow molding using at least one pressurized fluid, with or without stretching.

Different types of containers (bottles, flasks, jars, etc.) forming hollow bodies that are in particular, but not exclusively, designed to be used for the packaging of products in the food-processing industry are thus manufactured.

In the field of the manufacturing of containers for the food-processing industry, it is sought by any means to reduce the risks of microbiological contamination of the containers by pathogenic agents or microorganisms.

This is the reason for which the applicant already proposed to implement various actions to eliminate pathogenic agents, such as the germs (bacteria, molds, etc.) that are likely to affect the product that is contained in such containers.

The documents of the state of the art that are cited below, and to which reference will be made for more ample details, illustrate such actions by way of non-limiting examples.

It is possible in particular to distinguish, on the one hand, the actions whose purpose is to destroy the microorganisms to sterilize at least the inside of the container and, on the other hand, the actions whose purpose is more generally to prevent the contamination of the containers by such microorganisms.

The document FR-2,915,127 describes a container-manufacturing plant comprising a protective chamber that delimits a zone inside of which a blower-type container-molding machine is arranged, which machine is fed by means for transferring of preforms previously conditioned thermally in a furnace.

According to the teachings of this document, the plant comprises a system for blowing-in filtered air inside the chamber to establish therein in particular an overpressure in such a way as to limit the contamination risks of both preforms leaving the furnace as well as manufactured containers.

The document WO-03/084818 describes, for example, a decontamination treatment by irradiation of the neck of the preforms by an ultraviolet (UV)-type radiation, before the insertion of preforms into the furnace.

The document EP-2,094,312 describes, for example, a treatment by irradiation with an ultraviolet (UV) radiation that is implemented in a particular manner in a furnace for decontaminating at least the outer surface of the preform during thermal conditioning.

The document WO-2006/136498 describes, for example, a decontamination treatment of a preform that consists in depositing by condensation an essentially uniform vapor film of a sterilizing agent on the inner wall of the preform.

The decontamination of the preform is carried out there by means of a treatment device that intervenes before the insertion of the preform into the furnace.

Such a treatment is intended to destroy the pathogenic agents or microorganisms to decontaminate at least the inside of the preform corresponding to the so-called inner “food” surface of the container and will become one with it, i.e., the surface that, after filling, will be in direct contact with the product.

Note that the quantity of microorganisms may be identified by counting after, in particular, operations of washing, filtering, and cultivation.

A logarithmic reduction of the number of microorganisms, for example referred to as on the order of 3Log (or else 3D) equivalent to 1000 units (10³), is thus determined.

Such a treatment of decontamination by condensation, referred to as “chemical means,” is satisfactory since degrees of decontamination of up to 6Log are obtained.

However, alternative solutions that make it possible not to use a sterilizing agent, such as hydrogen peroxide (H₂O₂), are sought, and this so as to find solutions that are more environmentally friendly, but without, however, thereby sacrificing the result obtained for the decontamination.

The use of a sterilizing agent, such as hydrogen peroxide, requires the implementation of a set of particular means for meeting in particular regulatory obligations whose aim is to protect exposed individuals and more generally the environment (management of effluents, etc.), with this contributing to increasing the operating costs thereof.

Of course, the different examples of the above-mentioned actions are advantageously likely to be used in combination in the same plant for treating the different surfaces of a preform and more generally for reducing the risks of contamination in a drastic manner.

The containers made of thermoplastic material, such as the PET (PolyEthylene Terephthalate) in question here, are in particular but not exclusively bottles.

Such a hollow container is delimited as a whole by a wall and comprises a neck that radially delimits an opening and that extends by a body closed axially by a bottom.

For the sterilization of the inside of this type of container made of thermoplastic material, one of the problems encountered remains the limited accessibility to the inside surface of the container that presents the neck opening that in general has a reduced diameter.

One of the alternative solutions consists in using an ionizing radiation formed by an electron beam that is used to irradiate the surface to be sterilized.

The arrangement of an electron beam emitter outside of the container makes it possible to eliminate this problem of limited accessibility: the electrons of the beam emitted by the emitter radially pass through, from the outside to the inside, the wall of the body and the neck of said container to irradiate the inside of the container to be sterilized.

However, when the electron beam of the continuous type (in English “continuous e-beam”) that is used is emitted by a so-called “high-energy” emitter, i.e., in general with an energy of greater than 500 KeV and, for example, on the order of MeV, it is then noted that the electrons of such a continuous beam bring about modifications of the thermoplastic material with which said electrons interact by passing through the wall of the container.

However, such modifications alter the properties of the thermoplastic material of the container and are likely to compromise its subsequent use as packaging.

So as to limit the interactions between the continuous electron beam and the thermoplastic material, the use of a low-energy emitter (less than 500 KeV) has been considered.

However, the lower energy level of the electrons of the continuous beam is reflected by sterilization that is inadequate—starting from the moment when the beam is to pass through the wall of the container, of the body as of the neck—to succeed in irradiating its inside surface.

The desired degree of sterilization can therefore be obtained only by increasing the duration of irradiation for compensating for the weak penetration of the low-energy continuous electron beam but the durations necessary for the treatment of a container are then incompatible with the manufacturing rates.

In addition, problems of interactions between the continuous electron beam and the thermoplastic material remain, and the alteration of the thermoplastic material is all the greater the longer the duration of irradiation.

According to a known solution of the document U.S. Pat. No. 8,728,393, a portion of the problems can be resolved by introducing the continuous electron beam through the opening of the neck, directly inside, without passing through the wall.

Taking into account the neck diameters of a preform (or of a container), the emitter, however, remains outside of the preform, and the continuous electron beam is to be brought, guided to the inside, to be able to carry out the irradiation.

Such a solution is particularly complex to implement so that it can be used industrially and can irradiate the entire inner surface, which is the only solution that can ensure reliable sterilization.

By initiating the irradiation of the inside of the preform according to the document U.S. Pat. No. 8,728,393 and not that of the container obtained from such a preform, there is also a risk of contamination of the preform or of the container subsequent to the sterilization in such a way that, at the very least, drastic preventive measures should then be implemented to limit any risk of contamination subsequent to the sterilization by irradiation.

When the electron-beam guide means are inserted axially inside the preform for initiating the sterilization, there is then a risk of contamination of the inside of the preform, becoming a container.

Actually, such guide means are not sterile and can be the vector for microorganism contamination, in particular a contamination of the rim, i.e., of the circumferential edge of the neck that delimits the opening of the preform or of the container.

Although for the most part, the known solutions of the state of the art use a continuous-type electron beam, the document FR-2,861,215 also discloses the use of a pulsed-type, low-energy electron beam for the sterilization of packaging such as bottles.

As described in this document, the pulsed-type electron beam is obtained in particular by not applying in a permanent manner—but only for a given period—the voltage that brings about the acceleration of the electrons of the beam.

With an emitter such as the electron gun with focusing anode of the document FR-2,861,215, it is indicated by way of example that the voltage is applied for 2 μs (microseconds) with a frequency of 500 Hz, or an emission every 2 ms (milliseconds), and a current that has an intensity of 10 A.

The use of an electron gun with focusing anode according to the document FR-2,861,215 or an analogous emitter is not satisfactory, however, for sterilizing the inside of the containers made of thermoplastic material.

Actually, the treatment period that is necessary for irradiating the surface to be sterilized with a sufficient quantity of electrons, i.e., to obtain a lethal dose, is too long and is consequently not compatible with the current production rates of containers that, in the case of PET bottles, reach, for example, 50,000 to 60,000 bottles per hour.

With such a pulsed electron beam, the problems of altering the thermoplastic material also exist that no longer make it possible to consider the industrial application thereof.

The various alternative solutions known from the state of the art and that have just been described consequently are not satisfactory for sterilizing at least the inside of the containers made of thermoplastic material, in particular bottles made of PET.

The object of this invention is in particular to resolve at least a portion of the drawbacks of the state of the art and to propose a solution that makes it possible, without degrading its constituent material, to sterilize in a quick and reliable manner the inside of a container made of thermoplastic material.

For this purpose, the invention proposes a method for sterilizing containers made of thermoplastic material that comprises at least one step consisting in irradiating a container from the outside by means of a pulsed electron beam that is formed by a series of pulses, with each having a duration of emission of less than 100 ns and an intensity of greater than 1 kA to sterilize—through a wall of the container—the inside of said container.

Advantageously, the pulsed-type electron beam according to the invention is unobtrusive, formed by a series of pulses having a very high intensity on the order of a kiloampere (kA) and with a particularly short duration of emission on the order of a nanosecond (ns).

Owing to a pulsed electron beam having, in accordance with the invention, a duration of emission of less than 100 ns and an intensity of greater than 1 kA, the sterilization of the inside of the container is achieved, with not only a treatment period that is compatible with the manufacturing rates, but also and primarily without the interactions between the electrons of the pulsed beam and the thermoplastic material compromising the subsequent use of the sterilized container.

By comparison with a pulsed electron beam according to the document FR-2,861,215, the duration of emission of a pulse is very short since this duration is less than 100 ns in the case of the invention, for example several nanoseconds, whereas it is expressed in microseconds (μs) with an emitter such as the electron gun with focusing anode according to this document.

The pulsed electron beam according to the invention is obtained, for example, by means of an explosive electron emission, also sometimes referred to by the acronym E.E.E. for “Explosive Electron Emission” in English.

The brevity of a duration of emission of the pulsed electron beam on the order of a nanosecond combined with a high pulse intensity being expressed in kiloamperes limits the interactions of the electrons with the thermoplastic material while having an irradiation that makes possible effective sterilization.

The results that are obtained with the invention are particularly surprising especially as those obtained up until then, both with a continuous electron beam and with a pulsed electron beam (such as the one produced with an electron gun according to the document FR-2,861,215), discouraged one skilled in the art from continuing the sterilization of the inside of a container by means of an electron beam.

For one skilled in the art, it is consequently not possible, on the one hand, to sterilize the inside of a container quickly through its wall by means of an electron beam and, on the other hand, to do so without altering its thermoplastic material.

The use of an electron beam consequently goes against the prejudices of one skilled in the art. Actually, for one skilled in the art, it is not possible with an electron beam to sterilize the inside of a container by irradiating it from the outside through the wall without coming up against incompatible alterations of the thermoplastic material by the electron beam, as well as durations of irradiation for obtaining a lethal dose that are incompatible with an industrial application.

Advantageously, said pulsed electron beam is emitted with, between two successive pulses, a time interval (T) that is greater than 3 ms, for example equal to 10 ms.

Such a time interval advantageously participates in limiting the interactions of the electrons of the beam with the thermoplastic material.

In the absence of a substantial time interval in relation to the duration of emission of a pulse, the irradiation obtained with the pulsed electron beam ultimately differs little from that obtained with a continuous electron beam.

By comparison with the irradiation carried out with the solutions of the state of the art, the microorganisms are destroyed, surprisingly enough, with a greater effectiveness by a pulsed electron beam in accordance with the teachings of the invention.

Most particularly, the destruction of the microorganisms is more effective when the irradiation is carried out with a series of pulses that are very short and of high intensity (i) and advantageously a determined time interval (T) between two successive pulses.

Advantageously, the pulsed electron beam irradiates the microorganisms by alternating in a repeated manner “stress” moments of the microorganisms during which the latter are irradiated with a strong intensity, with a respite between two successive “stresses.”

Advantageously, said pulsed electron beam has energy, referred to as low energy, which is less than 500 KeV, preferably greater than 400 KeV.

Advantageously, the method comprises a step that consists in inserting a reflector axially inside the container that is to be sterilized.

Preferably, said step for inserting the reflector is carried out prior to the irradiation step. As a variant, the step for inserting the reflector is carried out during the irradiation step, in particular in such a way as to reduce the total period of treatment by irradiation to sterilize a container.

Advantageously, the reflector is sterilized by the pulsed electron beam according to the invention, with the outer surface of the reflector being at least irradiated by the electrons of the beam when the reflector extends axially inside the container.

Advantageously, the implementation of such a sterilization method is compatible with the container manufacturing rates and therefore able to accommodate an industrial application by integrating a device for sterilization within a plant for manufacturing containers such as bottles made of PET.

Advantageously, the sterilization of the container according to the method of the invention is carried out by irradiating an empty container, preferably just before initiating its filling.

By comparison with a method for sterilizing preforms by chemical means according to the above-mentioned document WO-2006/136498, the invention makes it possible to greatly simplify the design of a container manufacturing plant and to reduce its operating costs.

The sterilization of the final container (and not of the preform) advantageously makes it possible to eliminate the need for numerous means implemented until then in a plant for manufacturing containers starting from a preform, with the microorganisms that are present being destroyed during the irradiation of the container by means of the pulsed electron beam according to the invention.

Actually, by sterilizing the container and always by comparison, it is no longer necessary in particular to use specific means (such as blowing-in systems, etc.) to preserve the sterility of a preform after its treatment, i.e., during its thermal conditioning, its transformation by blow molding or stretch blow molding of the container, and this until the container is filled and closed.

Advantageously, the sterilization method according to the invention makes it possible to sterilize both the inside and the outside of the container.

The devices for treatment of the preforms by irradiation by means of UV radiation may, for example, be eliminated in the same way as the blowing-in systems and more generally filtration of the air participating in obtaining a suitable manufacturing environment.

Preferably, the sterilization method according to the invention is implemented in the container manufacturing plant between the molding unit (or blower) and the next unit, such as a filling unit.

The invention also proposes a sterilization device that comprises at least one emitter of a pulsed electron beam and an associated reflector inserted axially at least partially inside said container for selectively reflecting all or part of said pulsed electron beam emitted by said emitter from the outside, through a wall of the container, to irradiate said container so as to sterilize at least the inside of said container.

Advantageously, said sterilization device is intended to implement the method described above.

According to other characteristics of the sterilization device according to the invention:
The reflector is mounted to move axially, relative to the container, between at least a first position in which the reflector extends to the outside of the container and a second position in which the reflector, inserted through an opening that delimits a neck of the container, extends axially at least partially inside said container;
The reflector has a reflectance that varies axially, with said reflector comprising at least a first part that has a reflectance and a second part that has a reflectance that is less than the reflectance of the first part;
The first part of the reflector that has the reflectance and the second part of the reflector that has the reflectance are respectively manufactured from different materials;
The reflector comprises at least one specific part that has at least a reflective surface that does not extend in an axial plane;
   Said at least one specific reflective part is located at the free axial end of the reflector;
   Said at least one specific reflective part is formed by a ring extending radially projecting toward the outside;
   Said at least one specific reflective part comprises at least one tapered reflective surface;
The reflector comprises at least one part that has a determined electrical charge, with said charge being negative to obtain a repelling effect on the electrons or positive to obtain an absorption effect on the electrons;
   The device comprises means for driving the container in rotation to drive in rotation on itself said container relative to the emitter of the pulsed electron beam.

Other characteristics and advantages of the invention will emerge from reading the following description for the understanding of which reference will be made to the accompanying drawings in which:

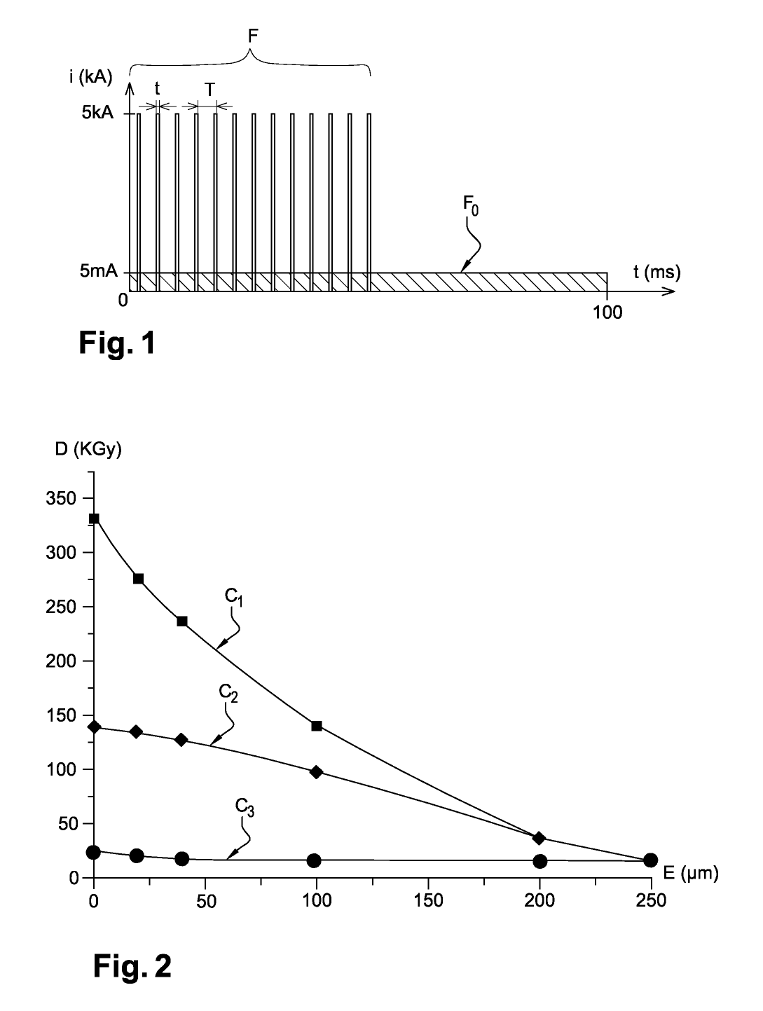

FIG. 1 is a graphic representation that shows on the ordinate the intensity (i) expressed in kiloamperes (kA) and on the abscissa the time expressed in milliseconds (ms) and that respectively illustrates a continuous electron beam F₀ and a pulsed electron beam F according to the invention that is formed by a series of pulses characterized by their duration (t) of emission, their intensity (i), and the frequency of emission of the pulses with an interval (T) between two pulses;

FIG. 2 is a graph that shows on the ordinate the dose (D) of electrons received expressed in kilograys (kGy) and on the abscissa the thickness (E) expressed in micrometers (μm) of the wall of a container made of PET and that illustrates the dose deposited on the outside surface of the container and through the wall to obtain a determined lethal dose on the inside surface of the container, with the curve C1 corresponding to an irradiation of the container with a continuous electron beam, with the curve C2 with a pulsed electron beam having an energy level of 250 KeV, and the curve C3 with a pulsed electron beam having an energy level of 430 KeV;

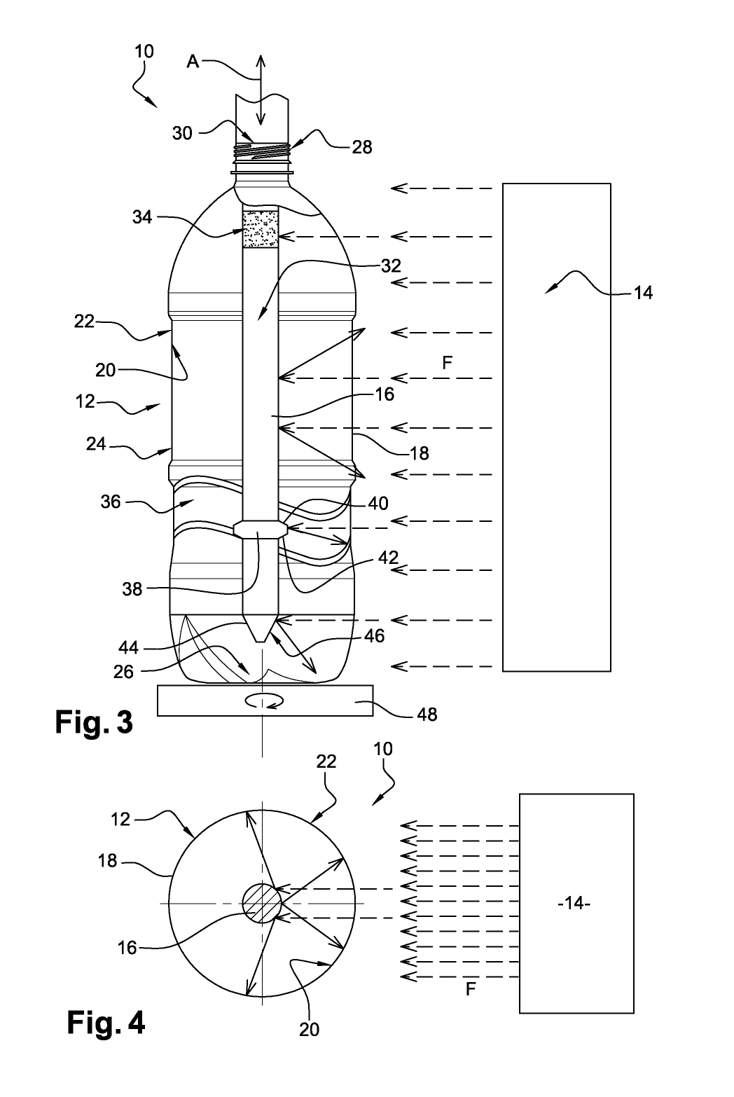

FIG. 3 is a side view that shows an embodiment of a sterilization device according to the invention and that illustrates the irradiation of a container by the sterilization device that comprises an emitter of a pulsed electron beam emitted radially from the outside and that is associated with a reflector that is axially inserted inside the container;

FIG. 4 is a top view that shows the container and the sterilization device according to FIG. 3 and that illustrates the sterilization of the container by the beam in accordance with the invention.

The method for sterilizing containers made of thermoplastic material according to the invention comprises at least one irradiation step that consists in irradiating a container from the outside by means of a pulsed electron beam (F) that is formed by a series of pulses, each having a duration (d) of emission of less than 100 ns and an intensity (i) of greater than 1 kA to sterilize—through a wall of the container—the inside of said container.

Advantageously, said pulsed electron beam (F) is emitted with, between two successive pulses, a time interval (T) of greater than 3 ms.

Advantageously, said pulsed electron beam (F) has energy, so-called low energy, of less than 500 KeV.

Preferably, said pulsed electron beam (F) has energy of greater than 400 KeV, for example on the order of 430 to 450 KeV.

As a variant, said pulsed electron beam (F) has energy, so-called low energy, of less than 500 KeV, which is, for example, equal to 250 KeV.

The graph of FIG. 1 shows a pulsed electron beam (F) according to the invention that is formed by a series of pulses, with the duration (t) of emission of each pulse being equal to 10 ns and with an intensity of 5 kA.

Said pulsed electron beam (F) of FIG. 1 has an energy level of 250 KeV, or a value of less than 500 KeV corresponding to a threshold value that is in general allowed between the “low energy” and the high energy.

Preferably, said pulsed electron beam (F) shown in FIG. 1 is emitted with, between two successive pulses, a time interval (T) that is equal to 10 ms.

For comparison purposes, FIG. 1 also shows a continuous electron beam (F₀) (cross-hatched) that is distinguished in particular from the pulsed electron beam (F) by the absence of a series of pulses between each of which the intensity (i) returns to a zero value.

The continuous electron beam (F₀) that is shown has the energy of 200 KeV, or low energy, and an intensity that is equal to 5 mA, with the duration of emission for initiating sterilization by irradiation being on the order of at least one second.

By comparison between these two types of electron beams, it is noted that the continuous electron beam requires a duration of emission of the beam for irradiating that is longer, and this for a minimum quantity of electrons received.

Actually, the pulsed electron beam consisting of the repetition of a series of very brief pulses makes it possible to irradiate the surface to be sterilized with a larger quantity of electrons, in particular because of the very great intensity of each pulse of the pulsed electron beam in relation to the intensity of the continuous electron beam.

The intensity of a pulse that is equal to 5 kA is very clearly greater than that of 5 mA of the continuous electron beam.

Thanks to this intensity on the order of kA, the number of electrons of the pulsed electron beam (F) that pass through the wall of the container for irradiating the microorganisms that are present inside the container will make it possible to sterilize both the outside and the inside of the container, and this over its entire height or axially from the neck to the bottom.

The irradiation obtained with a pulsed electron beam (F) is also effective on parts of the container having complex surfaces, for example because of the “design” of the container.

Thanks to a pulsed electron beam according to the invention, a lethal dose is applied to the inner surface of the container to be sterilized and this by transmitting lower energy to the thermoplastic material of the container through which this pulsed electron beam (F) passes, which advantageously limits the risks of alterations of the material but without sacrificing the effectiveness of the sterilization.

The irradiation of the microorganisms by a pulsed electron beam (F) is more effective because it is more difficult for the microorganisms to be protected from the repetition of pulses having the characteristics of duration (t) and intensity (i) of the pulsed electron beam (F).

Advantageously, the period of treatment with a pulsed electron beam (F) is less than that which would be necessary with a continuous electron beam (Fₒ) to obtain an irradiation by an equivalent quantity of electrons.

FIG. 2 is a graphic representation that illustrates the dose (D) expressed in kilograys (kGy) based on the thickness (E) in micrometers (μm) of the wall of a container made of PET, from the outer surface to the inside of the container to be sterilized.

The dose (D) in kilograys (kGy) corresponds to Joules per kg (kilogram), or energy per unit of volume, which, corresponding to a cumulative dose, illustrates the energy yielded by the electrons and absorbed by the material of the container.

The curve Cl corresponds to irradiation with a continuous electron beam (F₀); the curve C2 corresponds to irradiation with a pulsed electron beam (F₁) that has an energy level of 250 KeV; and the curve C3 corresponds to irradiation with a pulsed electron beam (F₂) that has an energy level of 430 KeV.

The value of 250 μm corresponds to a typical value for a wall of a container such as a bottle made of PET.

FIG. 2 shows the dose of radiation absorbed through a wall of 250 μm of PET with the various beams (F₀), (F₁), and (F₂) for obtaining—inside the container—a dose with a value that is at least equal to 14 kGy.

As illustrated in FIG. 2, the energy that is absorbed by the PET to obtain the desired lethal dose of at least 14 kGy at a depth of 250 μm is much lower when the electron beam is of the pulsed type in relation to a continuous electron beam (F₀), and by comparing the two beams of the pulsed type, the absorbed energy is still less with the beam (F₂) with the energy of 430 KeV than with the beam (F₁) with the energy of 250 KeV.

A beam (F) such as the pulsed electron beam (F₂) that has the energy of 430 KeV makes it possible to obtain a more homogeneous irradiation of the container through the wall, of the outside surface, and of the inside surface.

Advantageously, the energy absorbed by the PET is lower with such a beam (F₂), which reduces the risks of altering the thermoplastic material. The greater the energy of the beam, the greater will be the quantity of electrons passing through the wall of the container to irradiate its inside surface.

A beam (F₂) that has the energy of 430 KeV makes it possible, by comparison with the beam (F₁) with the energy of 250 KeV, to appreciably reduce the total duration of irradiation, which is particularly advantageous for an implementation in a container manufacturing plant.

Preferably, the beam (F) has energy of greater than 400 KeV.

To further improve the irradiation and to shorten the treatment time, the invention proposes associating with the emitter a reflector that is designed to be inserted axially inside the container through the opening of the neck so as to reflect the pulsed electron beam (F) selectively.

Advantageously, the method comprises, prior to the irradiation step, a step that consists in axially inserting a reflector inside of the container to be sterilized.

An embodiment of a device 10 for sterilizing a container 12 designed for the implementation of the sterilization method that was just described was shown in FIGS. 3 and 4.

In the description below, the “axial” orientation in reference to the main axis of the container and the direction of movement of the reflector as well as the “radial” orientation that is orthogonal to the “axial” orientation will conventionally be used in a non-limiting manner.

The device 10 for sterilizing containers 12 comprises at least one emitter 14 of a pulsed electron beam (F) and an associated reflector 16.

The reflector 16 is inserted axially at least partially inside said container 12 to reflect selectively all or part of said pulsed electron beam (F) emitted by said emitter 14 from the outside, radially through one wall 18 of the container, to irradiate said container 12.

To facilitate the representation of the beam by diffuse nature (electronic cloud), the electron beam (F) was shown in the form of a radial orientation arrow; however, such a representation is in no way limiting, with the rays of the beam (F) not being necessarily orthogonal to the axial direction.

The irradiation of the container 12 is more particularly designed to sterilize the inside of the container, i.e., the inner surface 20 of the container that will subsequently be in contact with a product, in particular a nutritional liquid such as water, milk, a juice, etc.

However, with the irradiation being carried out from the outside of the container 12 and through the wall 18, it will also sterilize the outer surface 22 thereof in such a way that the container 12 is sterilized in its entirety by the pulsed electron beam (F).

The container 12 shown in FIGS. 3 and 4 is only provided by way of example; the container 12 comprises a cylindrical body 24 that extends axially between a bottom 26 and a neck 28, with said neck 28 delimiting radially an opening 30.

The wall 18 has a given thickness (E) of thermoplastic material, for example of PET, and the term “wall” is to be understood in the broadest sense for the entire container 12, axially from the bottom 26 to the neck 28 and the body 24.

The reflector 16 is mounted to move axially, relative to the container 12, between at least a first position (not shown) and a second position shown in FIG. 3.

The first position corresponds to a position in which the reflector 16 extends outside of the container 12, totally apart from the container 12.

The first position is occupied in particular by the reflector 16 after the sterilization of a container 12 and while awaiting the sterilization of the next container 12.

The second position corresponds to a position in which the reflector 16, inserted through the opening 30 that the neck 28 of the container 12 delimits, extends axially at least partially inside said container 12.

Preferably, the reflector 16 is associated with drive means, such as an actuator, which is controlled to move axially, along the arrow A shown in FIG. 3, the reflector 16 relative to the container 12 occupying a stationary position.

As a variant, the reflector 16 could be stationary and the container 12 moved axially relative to the reflector 16 to insert the latter inside the container 12.

The reflector 16 is made in the form of an axial rod that has a maximum outer diameter that is less than the inner diameter of the neck of the container 12 in such a way as to be able to be inserted axially inside said container, preferably without contact with the neck 28 in particular.

Advantageously, the reflector 16 has a reflectance that varies axially according to the part of the reflector 16 being considered.

The reflector 16 comprises at least a first part 32 that has a reflectance R1 and a second part 34 that has a reflectance R2 that is less than the reflectance R1 of the first part 32.

Preferably, the second part 34 of the reflector 16 that has the reflectance R2 is located axially with respect to the reflector 16 and thus is located in the area of the neck 28 and/or the shoulder of the container 12 when the reflector 16 occupies said second position.

Advantageously, said at least one second part 34 of the reflector 16 that has the lower reflectance R2 is determined based on the “design” of the container 12, the part(s) of lesser reflectance such as the second part 34 being located axially on the reflector 16 and thus radially opposite the part(s) of the container 12 that are radially closer to the reflector 16, such as the shoulder of the container 12 that extends below the neck 28.

The reflector 16 is advantageously manufactured, in its entirety or partially, from at least one material that has a high relative atomic mass, preferably greater than 180, such as tantalum (Ta), tungsten (W), platinum (Pt) or gold (Au).

The first part 32 of the reflector 16 that has the reflectance R1 and the second part 34 of the reflector 16 that has the reflectance R2 are obtained, for example, by using different materials for each one.

As shown in FIGS. 3 and 4, the reflective outer surface of the reflector 16 is formed entirely or partially by a cylindrical surface that reflects with a given incidence the electrons of the beam F emitted in a pulsed manner by the emitter 14 radially through the wall 18 of the container 12.

The pulsed electron beam F according to the invention arrives orthogonally at the cylindrical surface of the reflector 16 before being reflected with a given incidence toward the inner surface 20 of the container 12 to be sterilized.

However, a container 12 such as a bottle made of PET in general has a particular “design” and thereby one or more zones, such as the specific zone 36 here in the shape of a wave, not having a cylindrical surface that extends axially parallel to that of the reflector 16 but comprising projecting and/or recessed portions.

To improve the irradiation of such specific zones, the reflector 16 comprises at least one specific part 38 in such a way as to reflect the pulsed electron beam F in the direction of at least one associated specific zone 36.

Advantageously, said specific part 38 of the reflector 16 comprises at least one reflective surface that does not extend in an axial plane.

In the example, said at least one reflective surface is neither parallel to the inner surface of the wall 18, nor orthogonal to the electron beam F emitted radially through the wall 18 of the container 12 by said emitter 14.

In the embodiment, said specific part 38 consists of at least one ring that extends radially projecting in relation to the rest of the reflector 16 and that in the axial cross-section has a profile in the shape of an elongated “V.”

The specific part 38 in the shape of a ring comprises an upper reflective surface 40 and a lower reflective surface 42, respectively tapered.

Advantageously, the reflector 16 comprises another specific reflective part 34 that is located at the free axial end of the reflector 16.

Preferably, said other specific reflective part 44 comprises at least one tapered reflective surface 46 designed to reflect the electron beam F in the direction of the bottom 26, in general of petaloid shape.

Advantageously, the reflector 16 comprises at least one part that has a determined electrical charge, with said charge being negative to achieve a repelling effect on the electrons of the beam F or positive to achieve an absorption effect on said electrons.

A variation of the reflectance according to the axial position of one given part of the reflector in relation to another may be obtained with parts that have different electrical charges.

Preferably, the reflector 16 is connected electrically to the ground or mass.

Advantageously, the device 10 comprises means 48 for driving in rotation the container 12 to drive it in rotation on itself relative to the emitter 14 of the pulsed electron beam F.

The sterilization device 10 that was just described constitutes the or one of the sterilization stations of a sterilization unit of a plant for manufacturing containers 12 made of thermoplastic material starting from hot preforms.

Such a unit for sterilizing containers 12 comprising at least one sterilization device 10 is arranged downstream from the molding unit in which the hot preforms, for example thermally conditioned in advance in a furnace, are transformed into containers 12 by blow molding or by stretch blow molding by means of at least one pressurized fluid.

Advantageously, the sterilization unit is arranged upstream from a unit for filling containers 12 that the plant for manufacturing containers 12 made of thermoplastic material comprises.

A plant for manufacturing containers 12 made of thermoplastic material of this type is known from the state of the art, and reference will be made to, for example, the document WO-99/03667, provided, however, in a non-limiting manner.

1. Method for sterilizing containers (12) made of thermoplastic material comprising at least one irradiation step consisting in irradiating a container (12) from the outside by means of a pulsed electron beam (F) that is formed by a series of pulses, each having a duration (d) of emission of less than 100 ns and an intensity (i) of greater than 1 kA to sterilize through a wall (18) of the container the inside (20) of said container (12).

2. Sterilization method according to claim 1, wherein said pulsed electron beam (F) is emitted with, between two successive pulses, a time interval (T) that is greater than 3 ms.

3. Sterilization method according to claim 1, wherein said pulsed electron beam (F) has energy, so-called low energy, of less than 500 KeV.

4. Sterilization method according to claim 1, wherein the method comprises a step that consists in axially inserting a reflector (16) inside of the container (12) to be sterilized.

5. Sterilization device (10) for the implementation of the sterilization method according to claim 1, wherein said sterilization device (10) comprises at least one emitter (14) of a pulsed electron beam (F) and an associated reflector (16) inserted axially at least partially inside said container (12) to reflect selectively all or part of said pulsed electron beam (F) emitted by said emitter (14) from the outside, through a wall (18) of the container, to irradiate said container (12) so as to sterilize at least the inside (20) of said container (12).

6. Sterilization device according to claim 5, wherein the reflector (16) is mounted to move axially, relative to the container (12), between at least a first position in which the reflector (16) extends outside of the container (12) and a second position in which the reflector (12), inserted through an opening (30) that delimits a neck (28) of the container, extends axially at least partially inside of said container (12).

7. Sterilization device according to claim 5, wherein the reflector (16) has a reflectance that varies axially, with said reflector (16) comprising at least a first part that has a reflectance (R1) and a second part that has a reflectance (R2) that is less than the reflectance (R1) of the first part.

8. Sterilization device according to claim 7, wherein the first part of the reflector (16) that has the reflectance (R1) and the second part of the reflector (16) that has the reflectance (R2) are respectively manufactured from different materials.

9. Sterilization device according to claim 5, wherein the reflector (16) comprises at least one specific part (38, 44) that has at least one reflective surface (40, 42, 46) that does not extend in an axial plane.

10. Sterilization device according to claim 5, wherein the reflector (16) comprises at least one part that has a determined electrical charge, with said charge being negative to achieve a repelling effect on the electrons or positive to achieve an absorption effect on said electrons.

11. Sterilization method according to claim 2, wherein said pulsed electron beam (F) has energy, so-called low energy, of less than 500 KeV.

12. Sterilization method according to claim 2, wherein the method comprises a step that consists in axially inserting a reflector (16) inside of the container (12) to be sterilized.

13. Sterilization method according to claim 3, wherein the method comprises a step that consists in axially inserting a reflector (16) inside of the container (12) to be sterilized.

14. Sterilization device (10) for the implementation of the sterilization method according to claim 2, wherein said sterilization device (10) comprises at least one emitter (14) of a pulsed electron beam (F) and an associated reflector (16) inserted axially at least partially inside said container (12) to reflect selectively all or part of said pulsed electron beam (F) emitted by said emitter (14) from the outside, through a wall (18) of the container, to irradiate said container (12) so as to sterilize at least the inside (20) of said container (12).

15. Sterilization device (10) for the implementation of the sterilization method according to claim 3, wherein said sterilization device (10) comprises at least one emitter (14) of a pulsed electron beam (F) and an associated reflector (16) inserted axially at least partially inside said container (12) to reflect selectively all or part of said pulsed electron beam (F) emitted by said emitter (14) from the outside, through a wall (18) of the container, to irradiate said container (12) so as to sterilize at least the inside (20) of said container (12).

16. Sterilization device (10) for the implementation of the sterilization method according to claim 4, wherein said sterilization device (10) comprises at least one emitter (14) of a pulsed electron beam (F) and an associated reflector (16) inserted axially at least partially inside said container (12) to reflect selectively all or part of said pulsed electron beam (F) emitted by said emitter (14) from the outside, through a wall (18) of the container, to irradiate said container (12) so as to sterilize at least the inside (20) of said container (12).

17. Sterilization device according to claim 6, wherein the reflector (16) has a reflectance that varies axially, with said reflector (16) comprising at least a first part that has a reflectance (R1) and a second part that has a reflectance (R2) that is less than the reflectance (R1) of the first part.

18. Sterilization device according to claim 6, wherein the reflector (16) comprises at least one specific part (38, 44) that has at least one reflective surface (40, 42, 46) that does not extend in an axial plane.

19. Sterilization device according to claim 7, wherein the reflector (16) comprises at least one specific part (38, 44) that has at least one reflective surface (40, 42, 46) that does not extend in an axial plane.

20. Sterilization device according to claim 8, wherein the reflector (16) comprises at least one specific part (38, 44) that has at least one reflective surface (40, 42, 46) that does not extend in an axial plane.
